(19) United States
(12) Patent Application Publication
(10) Pub. No.: US 2007/0218116 A1
(43) Pub. Date: Sep. 20, 2007

(54) Compositions and methods for the treatment of tumors and tumor metastases

(76) Inventor: Reto A. Schwendener, Kilchberg (CH)

Correspondence Address: HARNESS, DICKEY & PIERCE, P.L.C., P.O. BOX 8910, RESTON, VA, 20195, US

(21) Appl. No.: 11/374,039

(22) Filed: Mar. 14, 2006

(51) Int. Cl. A61K 9/127; A61K 31/675; A61K 31/663

(52) U.S. Cl. 424/450; 514/89; 514/102

(57) ABSTRACT

Compositions and methods for the treatment of tumors and tumor metastases Compositions and methods are provided to treat or prevent growth of tumors and tumor metastases, in particular of the following malignancies: breast cancer, cancer of the ovaries, cervical cancer, colorectal cancer, cancer of the prostate, lung cancer, cancer of the pancreas, kidney cancer, melanomas, malignant lymphomas and cancers of the central nervous system. The composition comprises a bisphosphonate and a liposome carrier, such as clodronate and a liposome carrier.

### FIELD OF INVENTION

The present invention relates to bisphosphonate compositions useful in the treatment of cancer and metastases.

### BACKGROUND

Bisphosphonates are compounds with a chemical structure similar to that of inorganic pyrophosphate (PPi), an endogenous regulator of bone mineralization. While PPi is comprised of two phosphate groups linked by phosphoanhydride bonds (a P—O—P structure), bisphosphonates are comprised of two phosphonate groups linked by phosphoether bonds to a central carbon atom (a P—C—P structure). Unlike the unstable nature of the P—O—P bonds, the P—C—P structure is highly resistant to hydrolysis under acidic conditions or by pyrophosphatases. Two additional covalent bonds to the central carbon atom of bisphosphonates can be formed with carbon, oxygen, halogen, sulfur, or nitrogen atoms, giving rise to a variety of possible structures. Like PPi, bisphosphonates form a three-dimensional structure capable of binding divalent metal ions such as Ca, Mg, and Fe in a bidentate manner, by coordination of one oxygen atom from each phosphonate group with the divalent cation. The affinity for calcium can be increased further if one of the side chains is a hydroxyl (—OH) or primary amino (—NH₂) group, because this allows the formation of a tridentate conformation that is able to bind Ca more effectively.

Two classes of bisphosphonates, the simple bisphosphonates (clodronate, etidronate) and the amino-bisphosphonates (tiludronate, alendronate, pamidronate, ibandronate, neridronate, risedronate, zoledronate and derivatives thereof) are known. The simple bisphosphonates are metabolized to non-hydrolizable analogs of adenosine triphosphate and diadenosine tetraphosphates (Rogers, M. J. et al., Biochem. Biophys. Res. Commun. 189: 414-423,1992; Frith, J. C. et al., J. Bone Miner. Res. 12: 1358-1367, 1997), whereas the amino-bisphosphonates are potent inhibitors of farnesyl diphosphate synthase, the major enzyme of the mevalonate pathway (Rogers, M. J. Calcified Tiss. Int., published online 31. Aug. 2004). Bisphosphonates are reviewed in: Fleisch, H., Endocr. Rev. 19: 80-100 (1998); Vasikaran, S. D., Ann. Clin. Biochem. 38: 608-623, 2001 and Ross, J. R. et al., Health Technology Assessment 8(4): A systematic review of the role of bisphosphonates in metastatic disease (2004), each of which is incorporated by reference in its entirety.

The high avidity of bisphosphonates for Ca ions is the basis of the bone-targeting property of these compounds. Bisphosphonates have therefore been widely used for treating osteolytic bone disease and osteoporosis, and to inhibit development of bone metastases or excessive bone resorption. It has been observed that bone metastasis, rheumatoid arthritis and osteoarthritis patients also experience decreased pain following bisphosphonate treatment (US patent application 20040176327).

US patent application 20040176327 discloses methods of treating angiogenesis by administering bisphosphonate to a patient, who may be a patient suffering from tumour growth or metastasis. Certain bisphosphonates are described as inhibiting growth factor induced angiogenesis but the inhibition appears not to depend upon depletion of activated macrophages but rather on activation or proliferation of endothelial cells. It has been observed that newly formed blood vessels resulting from angiogenesis disappear after bisphosphonate treatment, but that normal blood vessels remain intact (Giraudo, E. et al., J. Clin. Invest. 114: 623-633, 2004). Further it has been observed that the embolized blood vessels are not restored following cessation of the bisphosphonate treatment. Sebbah-Louriki et al. describe a new phenyl acetate bisphosphonate as inhibiting breast cancer cell growth in vitro. There is however no mention of treating angiogenesis or cancer using a bisphosphonate liposome formulation.

WO99/29345 relates to methods of inhibiting tumor growth by providing an environment free of activated macrophages and/or blocking the effects of factor(s) derived from activated macrophages which are necessary for angiogenic factors to cause angiogenesis in tumors. Although the use of certain diphosphonates with a suitable delivery vehicle, such as a liposome capsule agent, is mentioned for inhibiting the macrophage angiogenic effect through macrophage depletion, no supporting evidence is provided. It is presented that growth of certain tumor cell lines implanted in NOD/LtSz-scid/scid mice is inhibited due to the lack of activation of immature macrophages in this mouse model. An anti-M-CSF monoclonal antibody is also shown to suppress growth of murine tumors, as did a function-blocking anti-VEGF monoclonal antibody but no evidence is provided showing an effect on macrophage depletion.

US patent application 20030118637 discloses bisphosphonate compositions, which optionally include a liposome carrier, and their uses in treating autoimmune hemolytic anemia. Indeed, over twenty years ago it was found that incorporation of clodronate into liposomes allowed it to become a potent anti-macrophage agent both in vivo and in vitro (van Rooijen, N. et al., Cell Tissue Res. 238: 355-358, 1984; Seiler, P. et al., Eur. J.Immunol. 27: 2626-2633, 1997; van Rooijen, N. et al., J. Immunol Meth., 193: 93-99, 1996; van Rooijen, N. et al., J. Immunol. Meth. 174: 83-93, 1994). Thus, liposomal clodronate has been investigated as a potential therapeutic treatment for autoimmune disorders, such as adjuvant arthritis, uveitis, and experimental autoimmune encephalitis and other inflammatory diseases (Huitinga, I. et al., J. Exp. Med. 172: 1025-1033, 1990; Tran, E. H. et al., J. Immunol. 161: 3767-3775, 1998; Richards, P. J. et al., Rheumatology 38: 818-825, 1999; Madjdpour, C. et al., Am. J. Physiol. Lung Cell. Mol. Physiol. 284: L360-L367, 2003) and for various viral infection models (Seiler, P. et al., Eur. J. Immunol. 27: 2626-2633, 1997; Roscic-Mrkic, B. et al., J. Virol. 75: 3343-3351, 2001; Aichele, P. et al., J. Immunol. 171: 1148-1155, 2003).

There remains a need in the art for safer and more effective methods for the treatment or the prevention of the growth of tumors and/or tumor metastases and this invention meets those needs.

### RELEVANT LITERATURE

Alves-Rosa, F. et al., Blood. 96: 2834-2840, 2000
Berenson, J. R. et al. Semin. Oncol. 29(6 Suppl 21):12-18, 2002
Bingle, L. et al., J. Pathol. 196: 254-265, 2002
Conejo-Garcia, J. R., et al. Nat. Med. 10:950-958, 2004
Coukos, G., et al. Br. J. Cancer 92:1182-1187, 2005
Coussens, L. M. and Werb, Z., J. Exp. Med. 193: F23-F26, 2001
Coussens L. M. and Werb, Z., Nature 420(6917): 860-7, 2002
Dunn, G. P. et al., Nature Immunol. 3: 991-998, 2002
Dvorak, N. Engl. J. Med. 315: 1650-1659, 1986
Eubank, T. D. et al., J. Immunol. 171: 2637-2643, 2003
Eubank, T. D. et al., Immunity. 21: 831-842, 2004
Folkman, J., Curr. Mol. Med. 3: 643-651, 2003
Hagemann, T. et al. Carcinogenesis 25: 1543-1549, 2004
Khong, H. T. and Restifo, N. P., Nat. Immunol. 3: 999-1005, 2002
Lin, E. Y. and Pollard, J. W., Br. J. Cancer 90: 2053-2058, 2004
Mantovani, A. et al., FASEB J. 6: 2591-2599, 1992
Mantovani, A. et al., Trends Immunol. 23: 549-555, 2002
Mueller, M. M. and Fusenig, N. E., Nat. Rev. Cancer 4: 839-849, 2004
Mytar, B. et al, J. Leukoc. Biol. 74: 1094-1101, 2003
Pollard, J. W., Nat. Rev. Cancer 4: 71-78, 2004
Robinson, S. C. et al., Cancer Res. 63: 8360-8365, 2003
Rogers, M. J. et al., Cancer 88: 2961-2978, 2000
Seiler, P. et al., Eur. J. Immunol. 27: 2626-2633, 1997
van Rooijen, N. et al., Cell Tissue Res. 238: 355-358, 1984
van Rooijen, N. et al., J. Immunol. Meth. 174: 83-93, 1994
van Rooijen, N. et al., J. Immunol Meth., 193: 93-99, 1996
van Rooijen, N. et al., J. Leukoc. Biol., 62: 702-709, 1997
Vicari, A. P. and Caux, C., Cytokine Growth Factor Rev. 13: 143-154, 2002
Yamagishi, S. et al., Am. J. Pathol. 165: 1865-1874, 2004
Zou, W. Nat. Rev. Cancer 5:263-274, 2004

### BRIEF DESCRIPTION OF THE DRAWINGS

FIG. 1 is a graph showing that liposomal clodronate inhibits growth of F9 tumors in comparison to treatment with PBS as control when administered chronically, i.e. on days 0, 4, 8 and 12.

FIG. 2 is a graph showing that clodronate dissolved in PBS is not able to inhibit growth of F9 tumors as effectively as liposomal clodronate when administered chronically, i.e. on days 0, 4 and 8.

FIG. 3 is a graph showing growth curves of A673 tumors after treatment with clodronate-liposomes in comparison to treatment with PBS when administered chronically, i.e. on days 1, 5, 9 and 13.

FIG. 4 is a bar graph showing the body weight changes of A673 tumor bearing mice during clodronate-liposome therapy on days 1, 5, 9 and 13.

FIG. 5 is a bar graph showing that treatment with liposomal clodronate inhibits growth of F9 tumors more effectively than treatment with an anti-VEGF antibody and that the combination therapy of liposomal clodronate and an anti-VEGF antibody further increases the inhibition of tumor growth.

FIGS. 6A, B show that treatment with liposomal clodronate inhibits growth of A673 tumors more effectively than treatment with an anti-VEGF antibody and that the combination therapy of liposomal clodronate and an anti-VEGF antibody further increases the inhibition of tumor growth. FIG. 6A is a graph showing tumor growth (%) and FIG. 6B is a bar graph depicting tumor volumes measured five days after end of treatment (day 16).

FIG. 7 is a graph showing a dose-response curve of the toxicity of liposomal clodronate on cultured mouse peritoneal macrophages.

FIG. 8 is a bar graph showing that liposomal clodronate selectively inhibits the growth of macrophages in vitro without affecting the viability of other cell types.

### SUMMARY OF THE INVENTION

The present invention provides a method of treating or preventing tumor growth and/or metastasis in a patient, such as a human patient, comprising administering to a patient that has or is at risk of developing tumors and/or tumor metastases a composition comprising a bisphosphonate and a liposome carrier. The tumor can be breast cancer, ovarian cancer, gynecological cancer, hepatobiliary cancer, colorectal cancer, prostate cancer, lung cancer, pancreatic cancer, kidney cancer, bladder cancer, melanoma, malignant lymphoma and central nervous system cancer, head and neck cancer, or a tumor metastasis originating from any of the listed tumors. In one embodiment, the metastasis is not bone metastasis.

The bisphosphonate can be selected from the group consisting of clodronate (dichloromethylene diphosphonate), alendronate, etidronate, tiludronate, pamidronate, ibandronate, neridronate, zoledronate, minodronate and risedronate, and derivatives thereof, such as biologically active derivatives or prodrugs thereof. Preferably, the bisphosphonate is selected from the group consisting of clodronate (dichloromethylene diphosphonate), alendronate, tiludronate, pamidronate, ibandronate, neridronate, zoledronate, minodronate and risedronate, and biologically active derivative thereof, more preferably zoledronate or clodronate.

The liposome carrier preferably comprises cholesterol, phosphatidylcholine and/or tocopherol.

The composition can be administered to the patient intravenously, intraperitoneally, intraarterially, intratumorally, subcutaneously or orally, the route potentially depending on the type of tumor to be treated.

The bisphosphonate is typically administered in an amount sufficient to reduce the numbers of tumor associated macrophages and/or tumor metastases associated macrophages and/or tumor associated dendritic cells and/or tumor metastases associated dendritic cells in the patient by at least 10%, as compared to in the absence of said composition. The composition can be administered at intervals of from about 1 to 4 weeks, or at intervals of from about 1 to 7 days, depending on the dose, tumor stage or other parameter as will be apparent to the practitioner.

In another aspect of the invention, the method further incorporates the step of administring an anti-angiogenic drug, such as an anti-angiogenic antibody or antibody fragment thereof, e.g., an anti-VEGF antibody or anti-VEGF antibody fragment.

Optionally, the methods of the invention further comprise administering a second, different bisphosphonate and/or at least one cytotoxic drug.

In another aspect, the invention provides the use of a composition comprising a liposomal bisphosphonate for the manufacture of a medicament for the treatment and/or prevention of growth of tumors and/or tumor metastases, in particular, by depletion of tumor associated macrophages and/or tumor metastases associated macrophages and/or tumor and/or tumor metastases associated dendritic cells.

In a further aspect of the invention a composition for the treatment and/or prevention of growth of tumors and/or tumor metastases is provided comprising a bisphosphonate, optionally in a liposome formulation, and an anti-angiogenic drug and/or at least one cytotoxic drug. In some embodiments, the bisphosphonate is selected from the group consisting of clodronate (dichloromethylene diphosphonate), alendronate, etidronate, tiludronate, pamidronate, ibandronate, neridronate, zoledronate, minodronate and risedronate and derivatives thereof, and is preferably zoleondrate or clodronate (dichloromethylene diphosphonate), or derivatives thereof.

Also provided by the invention is a method of depleting tumor associated dendritic cells in a mammal, said method comprising administering a bisphosphonate to the mammal in a sufficient amount to reduce the number of tumor associated dendritic cells.

### DETAILED DESCRIPTION OF THE INVENTION

The term “depletion” as used herein with respect to tumor associated macrophages or tumor dendritic cells means a reduction in the number of these cells in a tumor relative to a control, such as the untreated tumor.

A liposomal bisphosphonate refers to a bisphosphonate encapsulated or incorporated within the trapped aqueous volume of a liposome or other composition comprising bisphosphonate and micelles or other lipid-containing vehicles, such as a complex or other mixture of lipid and a bisphosphonate, unless otherwise clear from the context.

In its broadest aspect, the present invention relates to a method to treat and/or prevent growth of tumors and/or tumor metastases comprising administering to a patient that has or is at risk of developing tumors and/or tumor metastases a composition comprising a bisphosphonate and a pharmaceutically acceptable carrier. The present inventor has found for the first time that liposomal bisphosphonates deplete specialized types of macrophages and phagocytic dendritic cells, namely tumor associated macrophages, tumor metastases associated macrophages, tumor associated dendritic cells and tumor metastases associated dendritic cells in the tumor mass. Accordingly, the present invention provides a method for the depletion of tumor associated macrophages and/or tumor metastases associated macrophages and/or tumor associated dendritic cells and/or tumor metastases associated dendritic cells in a patient in need of such treatment which comprises administering an effective amount of a bisphosphonate in a pharmaceutically acceptable composition to the patient.

The Examples below (Examples 1 and 2) illustrate the invention by demonstrating the inhibition of growth of tumors in vivo by administration of liposomal clodronate and furthermore, that liposomal clodronate is more effective than clodronate alone. However, it will be apparent that other bisphosphonates can be effective, combined with a pharmaceutically acceptable carrier such as a liposome. Bisphosphonates useful in the methods of the present invention include without limitiation clodronate (dichloromethylene diphosphonate), etidronate, tiludronate, alendronate, pamidronate, ibandronate, neridronate, risedronate, zoledronate, minodronate and their derivatives, including biologically active derivatives and prodrugs. In one embodiment, the bisphosphonate used in the present invention is clodronate or a derivative thereof. In another embodiment, the bisphosphonate used in the present invention is zoledronate or a derivative thereof. In yet another embodiment, the bisphosphonate used in the present invention is ibandronate or a derivative thereof.

Preferably, the bisphosphonate, when delivered to a macrophage, causes apoptosis and death of the macrophage. In a preferred embodiment, the bisphosphonate does not lead to the secretion of pro-inflammatory cytokines by the dying macrophages. In another embodiment, the bisphosphonate preferably acts on both macrophages and phagocytic dendritic cells. Most preferred is a bisphosphonate that has two or more of these three properties.

According to the present invention, a biologically active derivative, analog or prodrug of a bisphosphonate is any compound that is able to mimic the biological activity of a given bisphosphonate, often because the derivative has a basic structure that mimics the basic structure of the given bisphosphonate and/or has the salient biological properties of the given bisphosphonate compound. Biological activity can be measured using any suitable assay, including by evaluating any chemical or biological activity of the compound, such as those described in Fleisch, H., Endocrine Rev. 9: 80-100, 1998 and Ross, J. R. et al., Health Technology Assessment 8(4), 2004. Prodrugs may include biologically inactive derivatives, which are then metabolized by the body upon administration to provide a component with biological activity.

According to the present invention, the bisphosphonate is administered with a pharmaceutically acceptable carrier. As used herein, a pharmaceutically acceptable carrier refers to any substance or vehicle suitable for delivering a bisphosphonate useful in a method of the present invention to a suitable in vivo site. Such a carrier may allow the bisphosphonate component of the composition to come into contact with phagocytic cells, preferably tumor and/or tumor metastases associated macrophages, and/or tumor and/or tumor metastases associated dendritic cells in the patient and even more preferably, to be introduced into the phagocytic cells. Thus, pharmaceutically acceptable carriers can maintain a bisphosphonate in a form that, upon delivery of the bisphosphonate to a cell or tissue, the bisphosphonate contacts a cell of interest e.g., a phagocytic cell, such as a macrophage or a dendritic cell, and in addition enters the cell. In this way, the bisphosphonate may reduce the ability of the phagocytic cell to produce and shed chemoattractants, chemokines and angiogenesis promoting factors, including one or more of the following factors, basic fibroblast growth factor (bFGF), VEGF, angiopoietins (eg, ANG1 and ANG2), IL-1, IL-8, tumour necrosis factor-alpha (TNF-alpha), thymidine phosphorylase (TP), matrix metalloproteinases (e.g., MMP-9 and MMP-2), and nitric oxide (NO) or other factors. In particular, delivery allows the bisphosphonate to induce the phagocytic cell to undergo apoptosis and cell death. In one such embodiment, the invention includes the administration of a bisphosphonate (e.g., to a patient having a tumor and/or tumor metastases) as a liposomal bisphosphonate, such as a liposomal clodronate.

A pharmaceutically acceptable carrier can include a pharmaceutically acceptable excipient. Suitable excipients of the present invention include any excipients or formularies useful for in vivo delivery. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Alternatively, or in addition, aqueous carriers can contain cryoprotective agents that can preserve the structure of the liposomes and the concentration of liposomal bisphosphonate upon reconstitution of frozen and/or lyophilized compositions of bisphosphonate containing liposomes.

Thus, suitable pharmaceutically acceptable carriers for bisphosphonate compounds of the invention include, but are not limited to, liposomes, micelles or other lipid-containing vehicles, nanoparticles, nanovesicles, nanocontainers, microspheres, or solids which can be taken up in a suitable liquid as a suspension or solution (e.g., for injection), liquids that can be aerosolized, capsules or tablets. In a non-liquid formulation, the excipient can comprise, for example, dextrose, human serum albumin, and/or preservatives to which sterile water or saline can be added prior to administration (Moses, M. A. et al., Cancer Cell 4: 337-341, 2003).

Lipid-containing delivery vehicles include liposomes and micelles. A delivery vehicle can be modified to target to a particular site in a patient, thereby targeting and making use of the bisphosphonate at that specific site. Suitable modifications include manipulating the chemical formula of the lipid portion of the delivery vehicle and/or introducing into the vehicle a targeting agent (e.g., an antibody, antibody fragment, a polysugar or a peptide) capable of specifically targeting a delivery vehicle to a preferred site, for example, a preferred cell type (e.g., a tumor associated macrophage or dendritic cell). Examples of such cell type specific antibodies are referred to in Example 5.

A liposome delivery vehicle comprises a lipid composition that is capable of delivering a bisphosphonate or derivative thereof to a suitable cell and/or tissue in a patient. A liposome delivery vehicle comprises a lipid composition that is capable of fusing with the plasma membrane of the target cell (e.g., a phagocytic cell) to deliver the bisphosphonate or derivative thereof into a cell. As discussed above, liposome delivery vehicles can be modified to target a particular site in a mammal (i.e., a targeting liposome), thereby targeting and making use of a bisphosphonate or derivative thereof at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical (e.g. a charged peptide) can be added to the lipid formula of a liposome that alters the charge of the lipid bilayer of the liposome so that the liposome fuses with particular cells having particular charge characteristics. In one embodiment, other targeting mechanisms, such as targeting by addition of exogenous targeting molecules to a liposome (i.e., antibodies or peptides) may not be a necessary component of the liposome of the present invention, since effective delivery of the bisphosphonate can already be provided by the composition (e.g., if the bisphosphonate selectively acts on phagocytic cells, but not neutrophils or lymphocytes or other cells) without the aid of additional targeting mechanisms. However, in some embodiments, a liposome can be directed to a particular target cell or tissue by using a targeting agent, such as an antibody, antibody fragment, soluble receptor or ligand (e.g., a mannose residue), incorporated with the liposome, to target a particular cell or tissue to which the targeting molecule can bind. Targeting liposomes are described, for example, in Schwendener, R. A. et al., Biochim. Biophys. Acta 1026: 69-79, 1990; Marty, C. et al. Br. J. Cancer 87: 106-112, 2002; Marty, C. and Schwendener, R. A. Meth. Mol. Med. 109: 389-402, 2005 and U.S. Pat. No. 4,957,735 to Huang, L. et al., all of which are incorporated herein by reference in their entireties.

In one embodiment, if avoidance of the efficient uptake of liposomes by cells of the mononuclear phagocyte system due to opsonization of liposomes by plasma proteins or other factors is desired, hydrophilic lipids, such as gangliosides (Allen, T. M. et al., FEBS Lett. 223: 42-46, 1987) or polyethylene glycol (PEG)-derived lipids can be incorporated into the bilayer of a conventional liposome to form the so-called sterically-stabilized or “stealth” liposomes (Woodle M. C., Lasic, D. D., Biochim. Biophys. Acta. 1113: 171-199, 1992; Sapra P. and Allen T. M., Prog. Lipid Res. 42: 439-62, 2003; Allen, T. M. and Cullis, P. R., Science 303: 1818-1822, 2004). Variations of such liposomes are described, for example, in U.S. Pat. No. 5,705,187 to Unger et al., U.S. Pat. No. 5,820,873 to Choi et al., U.S. Pat. No. 5,817,856 to Tirosh et al.; U.S. Pat. No. 5,686,101 to Tagawa et al.; U.S. Pat. No. 5,043,164 to Huang et al., U.S. Pat. No. 5,013,556 to Woodle et al. and U.S. Pat. No. 6,660,525 to Martin et al. all of which are incorporated herein by reference in their entireties.

In another embodiment, where it is desired to optimize the pharmacokinetic properties and the organ distribution (in particular, to optimize distribution into a tumor and/or a tumor metastases) of a liposome, the liposome can be modified by methods known to one skilled in the art. In particular, a chemical can be added to the lipid formulation of a liposome to improve the pharmacokinetic property of the liposome so that the liposome is preferably distributed to the target tissue or organ, i.e. a tumor and/or tumor metastases. The above described sterically-stabilized liposomes are an example of such a modified liposome.

Suitable liposomes for use with the present invention include, but are not limited to, lipid compositions comprising phosphatidylcholine, dipalmitoylphosphatidylcholine, cholesterol, phosphatidylethanolamtine, phosphatidylserine, dimyristoyl- or distearoylphosphatidylcholine, other components, and combinations thereof. Preferred lipids are phosphatidylcholines and cholesterol and/or other components. The lipids can be provided in various forms, including, but not limited to, multilamellar vesicles (MLVs) and small unilamellar vesicles (SUVs) (Szoka, F. and Papahadjopoulos, D. Ann. Rev. Biophys. Bioeng. 9: 467-508, 1980; Mayer, L. D., et al. Chem. Phys. Lipids 40: 333-345, 1986; Milsmann, M. et al., Biochim. Biophys. Acta 512: 147-155, 1978), all of which are incorporated herein by reference in their entireties.

Encapsulating or incorporating a compound, such as a bisphosphonate, with a liposome is accomplished in a straightforward manner using techniques known in the art. The compound can be effectively encapsulated or incorporated within a liposome's trapped aqueous volume simply by gentle mixing of the compound and the liposome together, preferably in a suitable excipient. The compound can also be incorporated into the liposome as the liposome is formulated (e.g., rehydrated). For example, lipids can be dissolved in a solvent frozen and/or lyophilized, followed by rehydration in a liquid solution of a bisphosphonate. A suitable amount of liposome to use in preparing a composition can easily be determined by the skilled artisan for a given amount of bisphosphonate to be used.

A liposome delivery vehicle should be capable of remaining stable in a mammal for a sufficient amount of time to deliver a bisphosphonate and other compounds, if included, to a preferred site in the patient. A liposome carrier is preferably stable in the patient into whom it has been administered for at least 30 minutes, more preferably for at least 1 hour and even more preferably for at least 24-72 hours.

One type of pharmaceutically acceptable carrier includes a controlled release formulation that is capable of slowly releasing a composition of the present invention into a patient. As used herein, a controlled release formulation comprises a bisphosphonate or derivative thereof in a controlled release vehicle. Suitable controlled release vehicles include, but are not limited to, biocompatible polymers, other polymeric matrices, capsules, microcapsules, microparticles, nanoparticles, nanovesicles, nanocontainers; bolus preparations, osmotic pumps, diffusion devices, liposomes, lipospheres, and other delivery systems known to one skilled in the art.

In one aspect of the invention, the composition to be administered to the patient can include additional components that assist in the delivery of the bisphosphonate to the patient, that stabilize the bisphosphonate, or that provide an additional benefit to the patient that has, or is at risk of developing tumors and/or tumor metastases. For example, the composition can include a bisphosphonate and at least a second agent for the treatment or the prevention of the growth of tumors and/or tumor metastases, whereby the second agent is an anti-angiogenic drug, an anti-inflammatory drug, a cytotoxic drug, or combinations thereof. In some embodiments, such compositions will include a lipid/liposome vehicle as described above. Examples 3 and 4 illustrate this aspect of the invention by showing that liposomal clodronate is effective when given repeatedly and concurrently with an anti-angiogenic antibody (anti-VEGF scFv antibody fragment). Suitable anti-angiogenic drugs (e.g. Imatinib Mesylate (Gleevec; ST1571)), anti-inflammatory drugs and cytotoxic drugs (e.g., alkylating agents, nitrosoureas, antimetabolites, antitumor antibiotics, plant alkyloids, taxanes and hormonal agents) are well known in the art.

Alternatively, the second agent, such as an anti-angiogenic drug or a cytotoxic drug, need not necessarily be administered as a part of the same composition containing bisphosphonate, but can be administered in a separate composition at an appropriate time (including simultaneously) relative to the administration of bisphosphonate. Such a “chronic” or “metronomic” (separate and not simultaneous) administration is particularly amenable to allow liposomal bisphosphonate (e.g., liposomal clodronate) to be administered repeatedly over a long period of time at low doses. Indeed, liposomal bisphosphonate (e.g., liposomal clodronate) can be used intermittently as a tumor and/or tumor metastases associated macrophage and/or tumor and/or tumor metastases associated dendritic cell depleting agent in very slow growing solid tumors, for example in prostate cancer, thereby preventing the growth of a primary solid tumor and the escape and dissemination of primary tumor cells that form tumor metastases at organ sites distant from the location of the primary tumor.

Accordingly, the method of the present invention prevents growth of tumors and/or tumor metastases or treats tumors and/or tumor metastases in a patient such that the patient is protected from the growth or formation of tumors and/or tumor metastases. As used herein, the phrase “protected from a disease” (such as cancer) refers to reducing the symptoms of the disease, reducing the occurrence of the disease, and/or reducing the severity of the disease. Protecting a patient can refer to the ability of a therapeutic composition, when administered to a patient, to prevent a disease from occurring and/or to cure or to alleviate disease symptoms, signs or causes. As such, to protect a patient from a disease includes both preventing disease occurrence or recurrence (prophylactic treatment) and treating a patient that has a disease or that is experiencing initial symptoms or later stage symptoms of a disease (therapeutic treatment). In particular, protecting a patient from a disease is accomplished by reducing the ability of phagocytic cells in the patient, and particularly macrophages (i.e., tumor and/or tumor metastases associated macrophages and/or tumor and/or tumor metastases associated dendritic cells), to inhibit, reduce or abolish growth of pre-existing tumors and/or tumor metastases, and/or increasing the apoptosis of such cells in the patient. The term, “disease” refers to any deviation from the normal health of a patient and includes a state when disease symptoms are present, as well as conditions in which a deviation has occurred, but symptoms are not yet manifested.

More specifically, a composition as described herein, when administered to a patient by the method of the present invention, preferably produces a result which can include alleviation of the disease (e.g., reduction of at least one symptom or clinical manifestation of the disease), elimination of the disease, prevention of the disease, or alleviation of a secondary disease resulting from the occurrence of a primary disease.

According to the present invention, an effective administration protocol (i.e., administering a bisphosphonate composition of the invention in an effective manner) comprises suitable dose parameters and modes of administration that result in a reduction in the number of tumor and/or tumor metastases associated macrophages and/or tumor and/or tumor metastases associated dendritic cells from the patient, or an increase in the apoptosis of such tumor and/or tumor metastases associated macrophages, and/or tumor and/or tumor metastases associated dendritic cells in the patient that has or that may develop a tumor and/or tumor metastases, preferably so that the patient is protected from the disease (e.g., by disease prevention or prevention of disease recurrence, or by alleviating one or more symptoms of ongoing disease). Effective dose parameters can be determined using methods standard to the clinician. Such methods include, for example, determination of survival rates, side effects (e.g., toxicity) and progression or regression of disease.

In accordance with the present invention, a suitable single dose size is a dose that results in the above-identified responses in a patient when administered one or more times over a suitable time period. Doses can vary depending upon the patient (e.g., age, weight, sex, health, other symptoms), the form of the disease being treated (e.g., acute or chronic), the carrier used, and/or the route of administration. For example, in the treatment of acute disease, a suitable dose regimen can be a single dose, whereas in chronic patients, a suitable dose regimen can be multiple doses of smaller amount, at intervals of days or weeks. One of skill in the art can readily determine appropriate single dose sizes for a given patient based on the size of a patient and the route of administration. Typically, the effectiveness of the treatment will be monitored by measuring, for example, plasma levels of factors produced by tumor associated macrophages and/or tumor associated dendritic cells, e.g. VEGF and other factors released by tumor associated macrophages and/or tumor associated dendritic cells, or by diagnostic methods used for the detection of tumors and/or tumor metastases, allowing the treatment regimes to be adapted for a particular patient or patient group. Thus, dosage regimens may be adjusted to provide the optimum therapeutic response.

In one aspect of the invention, a suitable single dose of a composition of the present invention is an amount that, when administered by any route of administration, reduces phagocytic cells (and preferably tumor associated and/or tumor metastases associated macrophages, and/or tumor and/or tumor metastases associated dendritic cells) in a patient, as compared to a control, which might be a patient who has not been treated with the composition of the present invention, the patient prior to administration of the composition, or as compared to a standard established for the particular disease, patient type and composition.

A suitable dose is typically an amount that reduces the number of phagocytic cells (and preferably of tumor associated and/or tumor metastases associated macrophages and/or tumor and/or tumor metastases associated dendritic cells) in the patient, as compared to the control, by at least 10%, and more preferably at least 20%, and more preferably at least 30%, and more preferably at least 40%, and more preferably at least 50%, and more preferably at least 60%, and more preferably at least 70%, and more preferably at least 80%, and more preferably at least 90%, and more preferably at least 95% and even more preferably about 100%. The measurement of the effect of depletion of tumor associated macrophages and/or tumor associated dendritic cells in a patient can be performed using known methods (e.g., immunohistochemical analysis of a biopsy).

A therapeutically active amount of a pharmaceutical composition of the present invention means an amount effective at dosages and for periods of time necessary to achieve the desired result. As referred to above, this may also vary according to factors such as the disease state, age, sex, and weight of the subject. Bisphosphonates are known from previous studies to be non-toxic and well tolerated in mammals, including humans. A dose of around 5-100 mg/kg is likely a suitable initial dosage for a mammal and this dosage may be adjusted as required to provide a safe and effective amount. Thus, the dosage may be varied to 0.1 to 100 mg/kg, 0.5 to 30 mg/kg, or 1 to 5 mg/kg.

In yet another embodiment, the composition of the present invention is administered in a dose that is effective to reduce the number of tumor associated and/or tumor metastases associated macrophages in a patient within about 12, 24, 36, 48, 60, 72, 84, 96, 108, 120, 132, 144, 156 or 168 hours of administration of the composition, as compared to the control (or standard) as set forth above. Preferably, the composition is administered in a dose that is effective to reduce the number of phagocytic cells (and preferably of tumor associated and/or tumor metastases associated macrophages and/or tumor and/or tumor metastases associated dendritic cells) in the patient within the given time period by at least 10%, and more preferably at least 20%, and more preferably at least 30%, and more preferably at least 40%, and more preferably at least 50%, and more preferably at least 60%, and more preferably at least 70%, and more preferably at least 80%, and more preferably at least 90%, and more preferably at least 95%, and even more preferably about 100%.

As is apparent to one of skill in the art, the number of doses administered to a patient is dependent upon the extent of the disease and the response of an individual patient to the treatment. For example, an adult or a patient with chronic disease may require more doses or smaller or larger doses than a pediatric patient. Thus, it is within the scope of the present invention that a suitable number of doses includes any number required to treat a given disease and patient.

In order to achieve the desired effect at least one dose can be administered in acute disease, whereas in some patients at least 2 or more doses are administered (e.g., in chronic disease, multiple or continuous dosing may be necessary to control the disease). The typical interval between doses when more than one dose is required is at least 1 day, and more preferably at least 2 days, and more preferably at least 3 days, and more preferably at least 4 days, and more preferably at least 5 days, and more preferably at least 6 days, and more preferably at least 7 days, and more preferably at least 8 days, and more preferably at least 9 days, and more preferably at least 10 days, and more preferably at least 11 days, and more preferably at least 12 days and more preferably at least 13 days, and more preferably at least 14 days. In another embodiment, the interval between doses is at least 3-4 weeks. In another embodiment, the interval between doses is at least 4 weeks.

Administration of liposomal bisphosphonate (e.g., liposomal clodronate) can result in a temporary duration of action (e.g., 1-4 days for a single dose), a desirable property to avoid severe untoward toxic side-effects. Hence, liposomal bisphosphonate (e.g., liposomal clodronate) can be advantageously administered only once every week or once every 1-4 weeks.

As discussed above, a composition of the present invention is administered to a patient in a manner effective to deliver the composition to a cell, a tissue, and/or systemically to the patient, whereby a reduction or inhibition of the growth of a tumor and/or of tumor metastases by the depletion of tumor and/or tumor metastases associated macrophages is achieved as a result of the administration of the composition. Suitable administration protocols include any in vivo administration protocol. The preferred routes of administration will be apparent to those of skill in the art, depending on the patient, the bisphosphonate, the pharmaceutically acceptable carrier, and the specific type of neoplasia to be prevented or treated. Preferred methods of in vivo administration include, but are not limited to, intravenous administration, intraperitoneal administration, intradermal administration, intranodal administration, subcutaneous administration, intra-articular administration, intraventricular administration, oral administration, impregnation of a catheter, and direct injection into a tissue (e.g. a tumor and/or tumor metastases). Particularly preferred routes of administration include, intravenous, intratumoral, intraperitoneal, and subcutaneous administration. Combinations of routes of delivery can be used and in some instances, and may even enhance the therapeutic effects of the composition. It may be beneficial to complex the bisphosphonate or other agent to a carrier capable of withstanding degradation by digestive enzymes in the gut of a mammal for oral delivery. Examples of such carriers include plastic capsules or tablets, such as those known in the art.

One method of local administration is by direct injection. Direct injection techniques are particularly useful for administering a composition to a cell or tissue (e.g., a tumor and/or tumor metastases) that is accessible by surgery, and particularly, on or near the surface of the body. Administration of a composition locally within the area of a target cell refers to injecting the composition within centimeters and preferably, within millimeters from the target cell or tissue.

The composition and method of the present invention can be used to prevent and/or treat any patient that has, or is at risk of developing, any tumor and/or tumor metastases in which phagocytic cells (e.g., macrophages and/or dendritic cells) infiltrate or associate with the tumor and/or metastases tissue. In particular, the method and composition of the present invention is useful for treating and/or preventing neoplasias and include without limitation: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, malignant lymphomas and cancers of the central nervous system, cancer of the head and neck and tumor metastases originating from said tumors. In some embodiments, bone cancer and bone metastasis are not encompassed by the present invention.

Compositions of the invention can be administered to any member of the vertebrate class, mammalia, including, without limitation, primates, rodents, livestock and domestic pets, such as cats and dogs. Preferred mammals to protect are humans.

In one embodiment, the patient to be treated has undergone, is undergoing, or will undergo any of the conventional therapeutic treatments for neoplasias known in the art. For example, the patient can undergo anti-angiogenic treatment, either simultaneously or alternating with the bisphosphonate treatment of the invention. For example, a patient can receive anti-angiogenic treatment, and then a liposomal bisphosphonate treatment, and then, at an appropriate time (e.g. days, 1-4 weeks) after the liposomal bisphosphonate treatment, additional anti-angiogenic treatment. Protocols for such combination treatments can be determined based, for example, on the sensitivity to the patient, the responsiveness of the patient to one or the other treatment, and the overall improvement of the patient.

In another embodiment, the patient to be treated has undergone, is undergoing, or will undergo conventional therapeutic treatments for neoplasias. For example, the patient can undergo chemotherapeutic (cytotoxic) treatment, either simultaneously or alternating with the bisphosphonate treatment of the invention. A patient could thus receive chemotherapeutic (cytotoxic) treatment, and then a liposomal bisphosphonate treatment, and then, at an appropriate time (e.g. days, 1-4 weeks) after the liposomal. bisphosphonate treatment, additional chemotherapeutic (cytotoxic) treatment. Protocols for such combination treatment can be determined based, for example, on the sensitivity of the patient to the chemotherapeutic and/or combination therapy with liposomal bisphosphonate, the responsiveness of the patient to one or the other treatment, and the overall improvement of the patient.

The compositions described hereinabove are also useful to deplete tumor associated dendritic cells in a sample or in a mammal. A cellular sample may be treated by addition of a suitable amount of bisphosphonate, optionally added as a liposomal bisphosphonate. A mammal, in particular a human patient, may be treated by administering a bisphosphonate in a sufficient amount to reduce the number of tumor associated dendritic cells. In both cases, typically the amount used is sufficient to reduce the number of tumor and/or tumor metastases associated dendritic cells as compared to the control, by at least 10%, and more preferably at least 20%, and more preferably at least 30%, and more preferably at least 40%, and more preferably at least 50%, and more preferably at least 60%, and more preferably at least 70%, and more preferably at least 80%, and more preferably at least 90%, and more preferably at least 95% and even more preferably about 100%. Depletion of tumor associated dendritic cells can be determined using known methods (e.g., immunohistochemical analysis of a cell sample or a biopsy).

The following examples are provided for the purpose of illustration and are not intended to limit the scope of the present invention. The examples merely provide specific methodology useful in understanding and practice of the invention.

### EXAMPLES

### Example 1

### Liposomal Clodronate Inhibits Growth of F9 Tumors

This example illustrates that liposome clodronate preparations inhibit the growth of teratocarcinoma cells in a mouse model.

Clodronate liposomes were prepared essentially as previously described (Seiler, P. et al., Eur. J. Immunol. 27: 2626-2633, 1997) using commercially available clodronate (dichloromethylene diphosphonate). Briefly, for a preparation of 40 ml of clodronate liposomes the following composition was used. Soy phosphatidylcholine (SPC, 4 g, Epikuron 200, Lukas Meyer, Hamburg, Germany), cholesterol (0.6 g, Fluka, Buchs, Switzerland) and D,L-α-tocopherol (0.02 g, Merck, Darmstadt, Germany) corresponding to 1:0.3:0.01 mol parts, respectively, were prepared by freeze-thawing and filter extrusion as follows. The dry lipid mixture was solubilized in a physiologic phosphate buffer (20 mM, pH 7.4) supplemented with mannitol (230 mM) as cryoprotectant and 2.64 g clodronate (clodronic acid disodium salt tetrahydrate, CH₂Cl₂Na₂O₆P₂×4 H₂O, Mol. Wt. 361; Bioindustria L.I.M., Novi Ligure, Italy). The resulting multilamellar vesicles were freeze-thawed in 3-5 cycles of liquid nitrogen and water at 40° C., followed by repetitive (5-10×) filter extrusion through 400 nm membranes (Nuclepore, Sterico, Dietikon, Switzerland) using a Lipex™ extruder (Lipex Biomembranes, Inc., Vancouver, Canada).

For the determination of the liposome encapsulation efficiency of clodronate the preparations were trace labeled with ⁴⁵CaCl₂ (Amersham Pharmacia Biotech UK Ltd., Little Chalfont, UK) and 1 mM CaCl₂ as carrier.

Non-encapsulated clodronate was removed by dialysis (Spectrapore tube, 12-14'000 mol. wt. cut-off) with mannitol-phosphate (67 mM, pH 7.4) as dialysis buffer (1:100 v/v). All preparations were sterile filtered through a 0.45 μm or 0.2 μm filter (Gelman or Millipore) and aliquots of 1 ml were stored at −80° C. or lyophilized. Liposome size and homogeneity were routinely measured with a Nicomp laser light scattering particle sizer (Nicomp 370, Sta. Barbara, Calif.).

Under these conditions, liposomes typically containing approximately 20 mg/ml clodronate and having a mean diameter of 135±70 nm were prepared. It will however be recognized that alternative methods can be adapted to prepare liposomes with similar characteristics. Leakage of clodronate from the liposomes in aqueous suspension was less than 10% in 7 days.

Female Sv129 nude mice can be obtained commercially from Charles River Wiga (Sulzfeld, Germany). Exponentially growing F9 teratocarcinoma cells (6-8×10⁶/50 μl) were injected s.c. on the abdominal side of the Sv129 mice, which were kept in standard housing and with a normal diet. In some cases, the cells were mixed in a 1:1 v/v ratio with Matrigel (Beckton Dickinson, Basel, Switzerland) shortly before injection. Starting at different time points (e.g., 0, 4, 8 or 12 days) after tumor cell injection, the mice (6-8 per group, 20 g±10% body weight) were injected intraperitoneally (i.p.) with the clodronate-liposome preparation, 100 mg clodronate-liposome/kg body weight as initial dose, corresponding to 2 mg clodronate per mouse (20 g) followed by 50 mg/kg for the remaining doses or 1 mg clodronate per mouse. Clodronate was dissolved in PBS and given by the same dose and schedule as liposomal clodronate. Control mice were injected i.p. with PBS. Tumor growth was measured in a blinded fashion with a calliper every day and tumor volumes were calculated using the following equation: V=λab²/6 (a=largest tumor diameter, b=per-pendicular diameter). Mice were sacrificed 8-26 days after onset of treatment. Body weights were recorded daily.

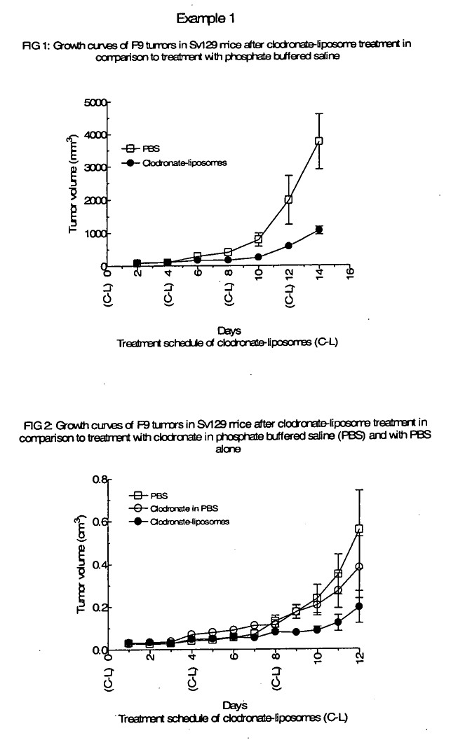

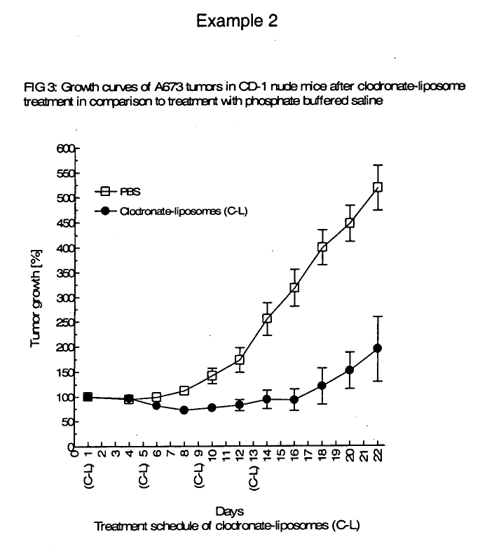

The results are depicted in FIG. 1 and FIG. 2, and show that liposomal clodronate inhibits growth of F9 tumors in vivo compared to PBS (FIG. 1), and that clodronate dissolved in PBS is not able to inhibit growth of F9 tumors as effectively as liposomal clodronate (FIG. 2). Tumor size reduction is quantified and provided in Table 1 below.

TABLE 1
tumor size reduction after various
treatments of F9 teratocarcinoma

| Treatment | Tumor volume (cm³) | Tumor size (%) |
|---|---|---|
| PBS | 3750 ± 852 | 100 |
| Clodronate liposome | 1055 ± 116 | 28 |
| Clodronate liposome + | 562 ± 205 | 15 |
| antibody | | |
| Antibody | 1708 ± 587 | 45 |
| Control Antibody | 3829 ± 171 | 102 |

### Example 2

### Liposomal Clodronate Inhibits Growth of A673 Tumors

This example illustrates that liposome clodronate preparations inhibit the growth of rhabdomyosarcoma cells in a mouse model.

In vivo testing of liposomal clodronate was carried out essentially as described in Example 1 but female CD1 nude mice (commercially available from Charles River Wiga, Sulzfeld, Germany) were used and exponentially growing human A673 rhabydomyosarcoma cells (6-8×10⁶/50 μl) were injected s.c. on the flanks of the CD1 mice.

The treatment of human A673 rhabdomyosarcoma bearing CD1 nude mice with clodronate-liposomes resulted in a significant inhibition of tumor growth as could be visualized macroscopically and further a visible reduction in blood vessels in the tumor mass could be ascertained.

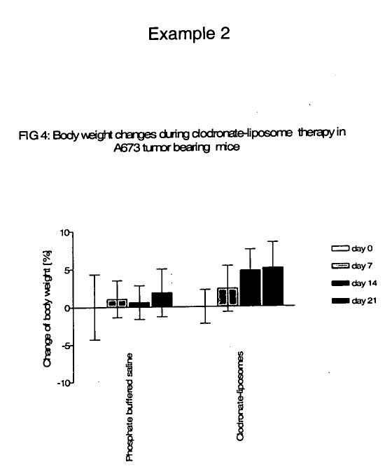

As depicted in FIG. 3 and Table 2 below, the treatment of human A673 rhabdomyosarcoma bearing CD1 nude mice with clodronate-liposomes resulted in a significant reduction of tumor growth. In this particular example, the onset of treatment was at an early stage of tumor development and the treatment was continued at an intermittent schedule of every 4 days. However it will be evident to the practitioner that similar results would be attainable with variations of this regime.

TABLE 2
tumor size reduction after various
treatments of A673 rhabdomyosarcoma

| Treatment | Tumor volume (cm3) | Tumor size (%) |
|---|---|---|
| PBS | 255 ± 33 | 100 |
| Clodronate liposome | 93 ± 19 | 36 |
| Clodronate liposome + | 53 ± 9 | 21 |
| antibody | | |
| Anibody | 110 ± 31 | 43 |
| Control Antibody | 259 ± 59 | 101 |

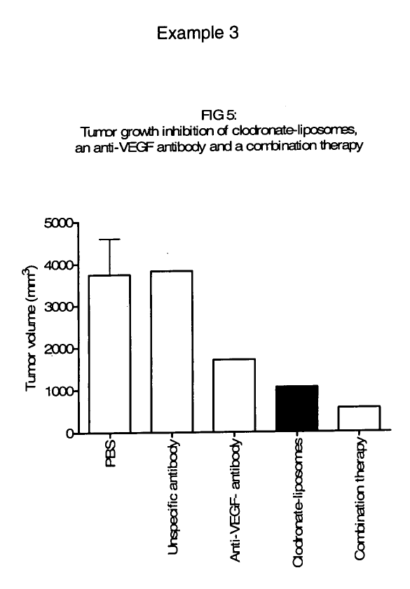

A severe side-effect of most anti-tumour compounds is body weight loss. Hence, the body weight of the treated mice was monitored and compared with controls. FIG. 4 is a bar graph showing the body weight changes of A673 tumor bearing mice during clodronate-liposome therapy on days 1, 5, 9 and 13. These data illustrate that treatment with clodronate-liposomes does not result in toxicity in vivo, as indicated by the treated mice maintaining body weight (i.e., not losing body weight).

### Example 3

### Additive Effects of Liposomal Clodronate and Anti-VEGF Antibody in Inhibiting Growth of F9 Tumors

This example shows that clodronate-liposome preparations inhibit teratocarcinoma tumor growth in vivo more effectively than treatment with an anti-VEGF antibody, and further that the combination therapy of liposomal clodronate and an anti-VEGF antibody potentiates the inhibitiory effect on tumor growth.

Anti-VEGF antibody was given by intravenous injection daily during 8 consecutive days (qd1-8) at a concentration of 50 mg scFv antibody/kg, alone or in combination with liposomal clodronate administered as described in Example 1. Liposomal clodronate was administered at the same dosage and time intervals as in Example 1. As can be seen in FIG. 5, clodronate-liposomes were more effective in inhibiting F9 tumor cell growth in vivo than an anti-angiogenic therapy with a VEGF-inhibiting scFv antibody, Furthermore, the combined administration of liposomal clodronate and an anti-angiogenic VEGF-inhibiting scFv antibody was most effective. Tumor size reduction is quantified and provided in Table 1 above.

### Example 4

### Additive Effects of Liposomal Clodronate and Anti-VEGF Antibody in Inhibiting Growth of A673 Tumors

This example shows that clodronate-liposome preparations inhibit rhabdomyosarcoma tumor growth in vivo more effectively than treatment with an anti-VEGF antibody, and further that the combination therapy of liposomal clodronate and an anti-VEGF antibody potentiates the inhibitiory effect on tumor growth.

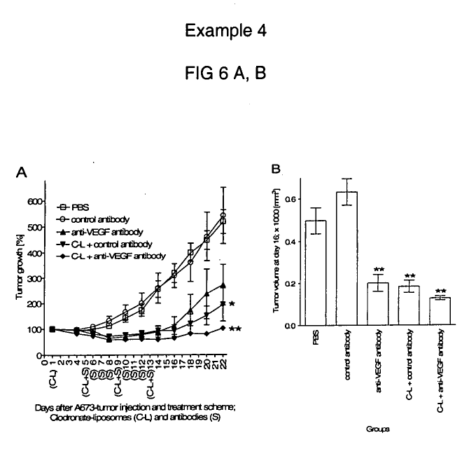

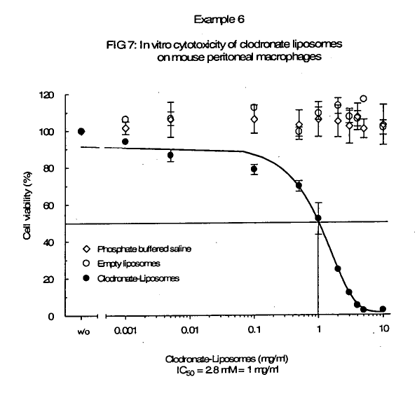

Anti-VEGF antibody was given by intravenous injection daily during 8 consecutive days (qd1-8) at a concentration of 50 mg scFv antibody/kg, alone or in combination with liposomal clodronate administered as described in Example 2. Liposomal clodronate was administered at the same dosage and time intervals as in Example 1. As can be seen in FIG. 6, clodronate-liposomes were more effective in inhibiting F9 tumor cell growth in vivo than an anti-angiogenic therapy with a VEGF-inhibiting scFv antibody. Furthermore, the combined administration of liposomal clodronate and an anti-angiogenic VEGF-inhibiting scFv antibody was most effective in inhibiting tumor cell growth. Tumor size reduction is quantified and provided in Table 2 above.

### Example 5

### Clodronate-Liposome Compositions Deplete TAMs and TADCs

This example shows that clodronate-liposome compositions deplete Tumour Associated Macrophages (TAMs) and Tumor Associated Dendritic Cells (TADCs).

Mice models of F9 teratocarcinomas or A673 rhabdomyosarcomas were treated with clodronate-liposome compositions essentially as described in Examples 1 and 2. Mouse tumor specimens were collected for histochemical analysis, immersed in Hanks balanced salt solution, and snap frozen in liquid nitrogen. Histological and immunohistochemical (IHC) assays were carried out as described by Mrkic, B., et al. (J. Virol. 74: 1364-1372, 2000). Briefly, two- to three-micrometer-thick tissue sections were cut in a cryostat, fixed with acetone, and stored at −70° C. For the staining of cell differentiation markers, the following primary rat anti-mouse monoclonal antibodies were used: antibodies against CD31 (CHEMICON International, Ltd.,United Kingdom), F4/80 macrophages (A3-1) (American Type Culture Collection, Manassas, Va.), marginal metallophilic or marginal zone macrophages (MOMA1 or ERTR9) (Biomedicals AG, Augst, Switzerland) and CD11 c (HL3) (PharMingen).

Primary antibodies were revealed by sequential incubation with alkaline phosphatase-labeled species-specific secondary antibodies (Jackson ImmunoResearch Labs, West Grove, Pa.). Alkaline phosphatase was visualized using naphthol AS-BI (6-bromo-2-hydroxy-3-naphtholic acid-2-methoxy anilide) phosphate and new fuchsin (Sigma) as a substrate, yielding a red reaction product. Sections were counterstained with hemalum.

Histological examination of tumors treated with liposomal clodronate using the macrophage-specific antibodies F 4/80 and MOMA1 showed depletion, i.e. a significant reduction of the number of tumor associated macrophages. In contrast, tumors from mice treated with an aqueous buffer solution (phosphate buffered saline, PBS) showed a significantly higher number of F 4/80 and MOMA1 stained tumor associated macrophages.

Surprisingly, tumor associated dendritic cells stained with the HL3 antibody, specific for CD11c positive dendritic cells were similarly depleted in tumors treated with liposomal clodronate.

In comparison to control tumors treated with PBS, treatment of tumors with liposomal clodronate also resulted in a significant reduction of blood vessel density in the tumors as demonstrated by staining of blood vessels with the endothelial cell specific CD31 antibody.

Thus, liposome clodronate depletes tumor associated macrophages and tumor associated dendritic cells.

### Example 6

### Liposomal Clodronate Selectively Inhibits the Growth of Macrophages In Vitro

This example illustrates that liposome clodronate selectively inhibits growth of macrophages in vitro without affecting the viability of other cells, an important test for evaluating cytotoxicity.

In brief, freshly isolated and 24-48 h cultured intraperitoneal macrophages, endothelial cells (PAE), and tumor cells (mouse F9 teratocarcinoma and human A673 rhabdomyosarcoma) were seeded at a density of 10⁴ per well in sterile 96 well plates in the presence of DME medium, supplemented with 10% FBS and antibiotics and cultivated for 48 h at 37° C., 5% CO₂. The cells were washed with PBS and incubated for 4, 6, and 12 h in the corresponding medium with clodronate dissolved in phosphate buffered saline (PBS), or with clodronate-liposomes with a final concentration of 0.00277-13.85 μM (0.001-5 mg/ml). The supernatants were removed and 100 μI/well of freshly diluted dye reagent solution in cell culture medium (1:10 v/v) (WST-1 dye reagent, Roche Diagnostics, Mannheim, Germany) was added and the mixture incubated for 60 min at 37° C. and 5% CO₂. Cell viability was determined by measuring the absorption at 410 nm using a Dynatech MR5000 plate reader (Microtec Produkte, Embrach, Switzerland).

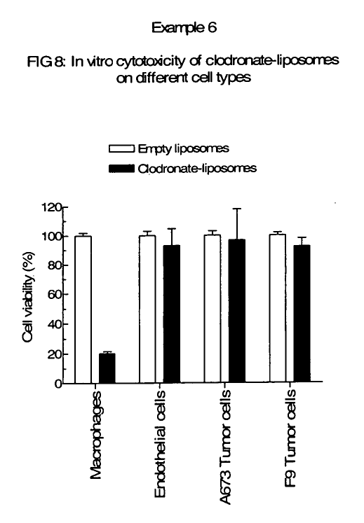

FIG. 7 depicts a graph showing a dose-response curve of the toxicity of liposomal clodronate on cultured mouse peritoneal macrophages. In vitro, liposomal clodronate was able to kill cultured macrophages, e.g. peritoneal mouse macrophages, whereas endothelial (e.g. porcine aortic endothelial cells, PAE) and tumor cells were not affected by the clodronate liposomes. The concentration of liposomal clodronate needed to inhibit growth of cultured macrophages in vitro was 2.8 mM FIG. 8.

In summary, the examples demonstrate that liposomal bisphosphonates can inhibit tumor growth. Although not wishing to be bound by theory, the present inventor believes that the advantageous effects are achieved through depletion of tumor associated macrophages (TAMs) and tumor associated dendritic cells (TADCs).

All publications and patent applications mentioned in this specification are herein incorporated by reference to the same extent as if each individual publication or patent application was specifically and individually indicated to be incorporated by reference. The invention now being fully described, it will be apparent to one of ordinary skill in the art that many changes and modifications can be made thereto without departing from the spirit or scope of the appended claims.

What is claimed is:

1. A method of treating or preventing tumor growth and/or metastasis in a patient, comprising administering to a patient that has or is at risk of developing tumors and/or tumor metastases a composition comprising a bisphosphonate and a liposome carrier.

2. The method of claim 1, wherein said tumor is breast cancer, ovarian cancer, gynecological cancer, hepatobiliary cancer, colorectal cancer, prostate cancer, lung cancer, pancreatic cancer, kidney cancer, bladder cancer, melanoma, malignant lymphoma and central nervous system cancer, head and neck cancer, or a tumor metastasis originating from said tumor.

3. The method of claim 1, wherein said metastasis is not bone metastasis.

4. The method of claim 1, wherein said patient is a human patient.

5. The method of claim 1, wherein said bisphosphonate is selected from the group consisting of clodronate (dichloromethylene diphosphonate), alendronate, etidronate, tiludronate, pamidronate, ibandronate, neridronate, zoledronate, minodronate and risedronate, and biologically active derivative thereof.

6. The method of claim 5, wherein said bisphosphonate is selected from the group consisting of clodronate (dichloromethylene diphosphonate), alendronate, tiludronate, pamidronate, ibandronate, neridronate, zoledronate, minodronate and risedronate, and biologically active derivative thereof.

7. The method of claim 5, wherein said bisphosphonate is clodronate (dichloromethylene diphosphonate) or a biologically active derivative thereof.

8. The method of claim 1, wherein said liposome carrier comprises cholesterol.

9. The method of claim 8, wherein said liposome carrier further comprises phosphatidylcholine and tocopherol.

10. The method of claim 1, wherein said composition is administered to said patient intravenously, intraperitoneally, intraarterially, intratumorally, subcutaneously or orally.

11. The method of claim 1, wherein said bisphosphonate is administered in an amount sufficient to reduce the numbers of tumor associated macrophages and/or tumor metastases associated macrophages and/or tumor associated dendritic cells and/or tumor metastases associated dendritic cells in said patient by at least 10%, as compared to in the absence of said composition.

12. The method of claim 1, wherein said composition is administered at intervals of from about 1 to 4 weeks.

13. The method of claim 1, wherein said composition is administered at intervals of from about 1 to 7 days.

14. The method of claim 1, wherein said method further comprises administration of an anti-angiogenic drug.

15. The method of claim 14, wherein said drug is an anti-angiogenic antibody or antibody fragment thereof.

16. The method of claim 15, wherein said drug is an anti-VEGF antibody or anti-VEGF antibody fragment.

16. The method of claim 1, further comprising administering a second, different bisphosphonate.

17. The method of claim 1, wherein said method further comprises administering at least one cytotoxic drug.

18. A composition for the treatment and/or prevention of growth of tumors and/or tumor metastases comprising a bisphosphonate and an anti-angiogenic drug.

19. The composition of claim 18, wherein said bisphosphonate is a liposomal bisphosphonate.

20. A composition for the treatment and/or prevention of growth of tumors and/or tumor metastases comprising a bisphosphonate and at least one cytotoxic drug.

21. The composition of claim 20, wherein said bisphosphonate is a liposomal bisphosphonate.

22. The composition of claim 20, wherein said bisphosphonate is selected from the group consisting of clodronate (dichloromethylene diphosphonate), alendronate, etidronate, tiludronate, pamidronate, ibandronate, neridronate, zoledronate, minodronate and risedronate and derivatives thereof.

23. The composition of claim 20, wherein said bisphosphonate is clodronate (dichloromethylene diphosphonate) or a derivative thereof.

24. A method of depleting tumor associated dendritic cells in a mammal, said method comprising administering a bisphosphonate in a sufficient amount to reduce the number of tumor associated dendritic cells.
